(19) United States
(12) Patent Application Publication
(10) Pub. No.: US 2026/0232873 A1
(43) Pub. Date: Aug. 13, 2026

(54) SURGICAL INSTRUMENT CABLE COATINGS AND SYSTEMS AND METHOD FOR MAKING THE SAME

(71) Applicant: Covidien LP, Mansfield, MA (US)

(72) Inventors: Michael A. Zemlok, Prospect, CT (US); David M. Chowaniec, Rocky Hill, CT (US); Matthew S. Hartzsch, Glastonbury, CT (US)

(21) Appl. No.: 19/154,242

(22) PCT Filed: Feb. 6, 2024
(86) PCT No.: PCT/IB2024/051096

§ 371 Date: Aug. 6, 2025

Related U.S. Application Data
(60) Provisional application No. 63/445,048, filed on Feb. 13, 2023.

(51) Int. Cl. A61L 31/08; A61B 34/00; A61L 31/10; A61L 31/14

(52) U.S. Cl. CPC A61L 31/088; A61B 34/71; A61L 31/10; A61L 31/14

(57) ABSTRACT

A surgical instrument includes a pulley rotatable about a pivot pin and an end effector coupled to the pivot pin. The instrument also includes a cable wound at least partially around the pulley and configured to actuate the end effector. The cable and/or any of the pulley components (e.g., the pulley, the pivot pin, etc.) includes a first coating having tungsten disulfide.

### CROSS-REFERENCE TO RELATED APPLICATION

This application claims the benefit of U.S. Provisional Patent Application Ser. No. 63/445,048, filed Feb. 13, 2023, the entire content of which is incorporated herein by reference.

### BACKGROUND

Surgical instruments utilize a variety of mechanisms to actuate and articulate end effectors. With the advent of surgical robotic systems and handheld powered instruments, cable-actuated mechanisms have become more prevalent. However, the cables are prone to failure due to being pulled at high loads over pulleys and other mechanisms that are used to constrain and redirect the cables during manipulation. Thus, localized wear of cables occurs due to high pressures between sliding surfaces within the twisted cable wire filaments as well as between the cable and pulley surfaces. Conventional lubricants are removed during sterilization processes, which may involve steam, solvents, high temperatures, etc. Considering that the instruments undergo multiple uses and sterilizations, the amount of the lubricant on the cables decreases with every use and sterilization. Thus, there is a need for a lubricated cable suitable for use in a robotic or powered surgical instruments that is capable of withstanding one or more sterilization cycles.

### SUMMARY

The present disclosure provides coatings and methods of forming coatings on cables used in surgical instruments. The cables may be multi-filament cables formed from any suitable metal, such as tungsten. The coatings lubricate the cables as well as individual filaments thereby reducing frictional wear, increasing the hardness and the galling of wear surfaces. The coatings also provide resistance to corrosion and chemicals, which allows for the surgical instrument to undergo multiple reprocessing cycles.

According to one embodiment of the present disclosure, a surgical instrument is disclosed. The surgical instrument includes a pulley rotatable about a pivot pin and an end effector coupled to the pivot pin. The instrument also includes a cable wound at least partially around the pulley and configured to actuate the end effector. The cable includes a first coating having tungsten disulfide.

Implementations of the above embodiment may include one or more of the following features. According to one aspect of the above embodiment, the first coating has a hardness of about 35 on a Rockwell C hardness (HRC) scale. The first coating may be disposed over a higher hardness base coating coating having a hardness of larger than 35 HRC. The harder base coating is configured to improve the surface wear attributes of the cable.

The harder base coating may include, but is not limited to, at least one of the following materials: titanium nitride, titanium, nickel, nickel boron, nickel-polytetrafluoroethylene, chromium, chromium nitride, chromium nickel, zirconium nitride, amorphous carbon, boron, boron carbide, molybdenum disulfide, molybdenum, graphite, silicone, or zirconium oxide. The first coating may have a static coefficient of friction of about 0.6 or less and a dynamic coefficient of friction of about 0.3 or less. The first coating may have a homogenous thickness from about 0.0005 mm to about 0.05 mm. The cable may further include a second coating disposed over the first coating. The second coating may include a polymer, which may include, but is not limited to, at least one of the following: polytetrafluoroethylenes, tetrafluoroethylenes, perfluoroalkoxy alkanes, fluorinated ethylene propylenes, polyimides, polyethylenimines, polyoxymethylenes, polyether ether ketones, copolymers, or combinations thereof. The cable may further include a third coating disposed over the second coating. The third coating may include at least one or more of the following: a wax-based, a paraffin-based, a grease-based, or an oil-based lubricant, which may include, but is not limited to, at least one of the following: graphite, petroleum, PTFE, TFE, molybdenum, polyalphaolefin (PAO), polyalkylene glycol (PAG), and/or silicone. All of the material coating types and combinations may also be used and incorporated onto any portion of the pulley components.

According to another embodiment of the present disclosure, a system for coating a surgical instrument cable is disclosed. The system includes a mold having a first portion and a second portion defining a mold cavity configured to receive the surgical instrument cable. The mold cavity may include a plurality of detents running along a length of the mold cavity. The system also includes a source of a liquid material for forming a coating on the surgical instrument cable inside the mold cavity, and one or more gates configured to distribute the liquid material through the mold cavity to form a smooth, thin, homogenous coating on the surgical instrument cable.

Implementations of the above embodiment may include one or more of the following features. According to one aspect of the above embodiment, each detent of the plurality of detents may be disposed in a parallel configuration relative to other detents along the length of the mold cavity. Each detent of the plurality of detents may be also disposed in a spiral configuration along the length of the mold cavity to assist the mold flow and mold application process. The first portion and the second portion of the mold may contact each other along a mold parting line and the mold cavity may further include a first pair of channels disposed along the mold parting line and a second pair of channels disposed in a plane perpendicular to the mold parting line.

### BRIEF DESCRIPTION OF THE DRAWINGS

Various embodiments of the present disclosure are described herein with reference to the drawings wherein:

FIG. 1 is a side view of a cable according to the present disclosure;

FIG. 2 is a transverse, cross-sectional view of the cable of FIG. 1;

FIG. 3 a perspective view, with parts separated, of an instrument drive unit and a surgical instrument according to an embodiment of the present disclosure;

FIG. 4 is a top, perspective view of an end effector of the surgical instrument of FIG. 3 according to an embodiment of the present disclosure;

FIG. 5 is a transverse, cross-sectional diagram of the cable of FIG. 1 and an enlarged view of the same according to an embodiment of the present disclosure;

FIG. 6 is a schematic diagram of an injection mold gating manifold depicting one or more gates for over-molding cable of FIG. 1 according to an embodiment of the present disclosure;

FIG. 7 is a transverse, cross-sectional view of a mold of the coating system of FIG. 4 according to one embodiment of the present disclosure;

FIG. 8 is a transverse, cross-sectional view of a mold of the coating system of FIG. 4 according to another embodiment of the present disclosure; and

FIG. 9 is a flow chart of a method for coating the cable of FIG. 1 according to one embodiment of the present disclosure.

### DETAILED DESCRIPTION

Embodiments of the presently disclosure are described in detail with reference to the drawings, in which like reference numerals designate identical or corresponding elements in each of the several views.

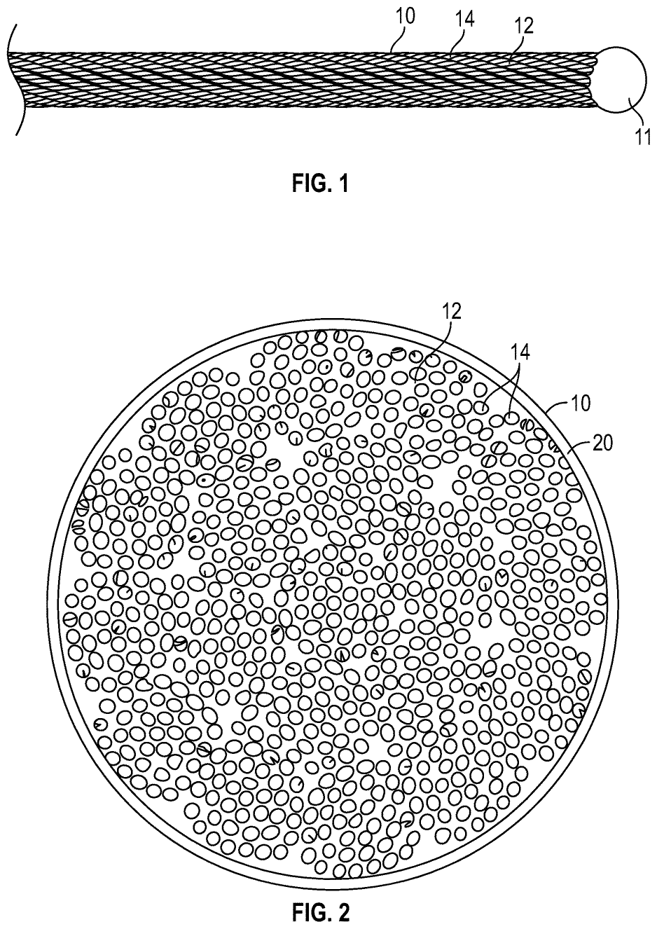

The present disclosure provides cables for use with surgical instruments, in particular surgical robotic and powered instruments that apply high loads to the cables actuating and articulating end effectors of the surgical instruments. As shown in FIGS. 1 and 2, a cable 10 is a multi-filament cable and includes a plurality of individual filaments 14 that are braided into a plurality of bundles 12, each including a plurality of filaments 14. The bundles 12 may be formed by braiding, twisting, etc. of the filaments 14 and the cable 10 may, in turn, be formed by braiding, twisting, etc. the bundles 12. The cable 10 may also include a ferrule 11 or a crimped end to secure bundles 12 and the filaments 14.

The bundles 12 may include any suitable number of filaments 14, which may be from about 6 to about 40. The bundles 12 may include the same different number of filaments 14 as shown in FIG. 1, which shows a 19×19 cable 10 (nineteen bundles 12 each having nineteen filaments 14). In embodiments, some of the bundles 12 may include varying number of filaments 14 and the bundles 12 may be arranged in any suitable manner such that the bundles 12 with fewer number of filaments 14 may be used to form a core of the cable 10 with the larger bundles 12 being braided around the central core. The filaments 14 may be drawn from any suitable metal, such as tungsten, stainless steel, and the like. The filaments 14 may have a diameter from about 0.001 mm to about 0.002 mm. The cable 10 may have a diameter of from about 0.4 mm to about 1 mm depending on the number of filaments 14, bundles 12, their respective diameters, and configurations described above.

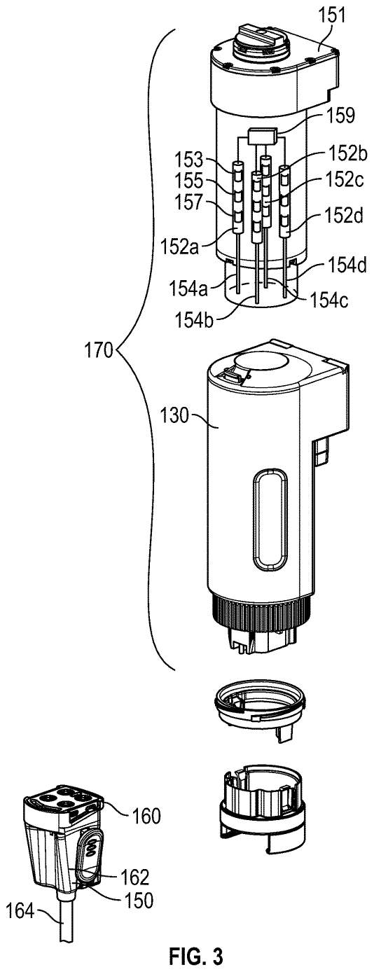

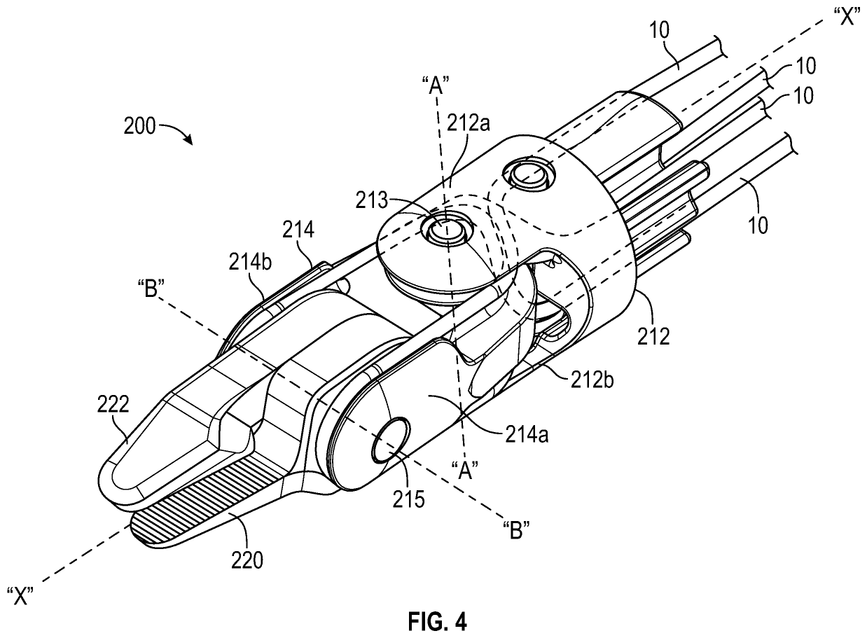

The cables 10 according to the present disclosure may be used in any cable-actuated surgical instrument, such as an instrument 150 of FIGS. 3 and 4. With reference to FIG. 3, the instrument 150 is actuated by an instrument drive unit (IDU) 170, which is configured to transfer power and actuation forces from its motors 152a, 152b, 152c, 152d to the instrument 150 to drive the instrument 150, such as articulation, rotation, pitch, yaw, clamping, cutting, etc. The IDU 170 may be disposed on a robotic arm or a handheld platform. The IDU 170 may also be configured for the activation of various functions of the instrument 150, such as ejection of staples, advancing a knife, supplying electrosurgical energy, etc.

The IDU 170 includes a motor pack 151 and a sterile barrier housing 130. Motor pack 151 includes motors 152a, 152b, 152c, 152d for controlling various operations of the instrument 150. The instrument 150 is removably couplable to IDU 170. As the motors 152a, 152b, 152c, 152d of the motor pack 151 are actuated, rotation of the drive transfer shafts 154a, 154b, 154c, 154d of the motors 152a, 152b, 152c, 152d, respectively, is transferred to the drive assemblies of the instrument 150. The instrument 150 is configured to transfer rotational forces/movement supplied by the IDU 170 (e.g., via the motors 152a, 152b, 152c, 152d of the motor pack 151) into longitudinal movement or translation of the cables or drive shafts to effect various functions of an end effector 200 (FIG. 4).

Each of the motors 152a, 152b, 152c, 152d includes a current sensor 153, a torque sensor 155, and an encoder sensor 157. For conciseness, only operation of the motor 152a is described below. The sensors 153, 155, 157 monitor the performance of the motor 152a. The current sensor 153 is configured to measure the current draw of the motor 152a and the torque sensor 155 is configured to measure motor torque. The torque sensor 155 may be any force or strain sensor including one or more strain gauges configured to convert mechanical forces and/or strain into a sensor signal indicative of the torque output by the motor 152a. The encoder sensor 157 may be any device that provides a sensor signal indicative of the number of rotations of the motor 152a, such as a mechanical encoder or an optical encoder. Parameters which are measured and/or determined by the encoder sensor 157 may include speed, distance, revolutions per minute, position, and the like. The sensor signals from sensors 153, 155, 157 are transmitted to the IDU 170, which then controls the motors 152a, 152b, 152c, 152d based on the sensor signals. In particular, the motors 152a, 152b, 152c, 152d are controlled by an actuator controller 159, which controls torque outputted and angular velocity of the motors 152a, 152b, 152c, 152d. In embodiments, additional position sensors may also be used, which include, but are not limited to, potentiometers coupled to movable components and configured to detect travel distances, Hall Effect sensors, accelerometers, and gyroscopes.

With reference to FIG. 3, instrument 150 includes an adapter 160 having a housing 162 at a proximal end portion thereof and an elongated shaft 164 that extends distally from housing 162. Housing 162 of instrument 150 is configured to selectively couple to IDU 170 of robotic, to enable motors 152a, 152b, 152c, 152d of IDU 170 to operate the end effector 200 of the instrument 150. Housing 162 of instrument 150 supports a drive assembly that mechanically and/or electrically cooperates with motors 152a, 152b, 152c, 152d of IDU 170. Drive assembly of instrument 150 may include any suitable electrical and/or mechanical component to effectuate driving force/movement.

The surgical instrument also includes an end effector 200 coupled to the elongated shaft 164. The end effector 200 may include any number of degrees of freedom allowing the end effector 200 to articulate, pivot, etc., relative to the elongated shaft 164. The end effector 200 may be any suitable surgical end effector configured to treat tissue, such as a dissector, grasper, sealer, stapler, etc. As shown in FIG. 4, the end effector 200 may include a pair of opposing jaws 220 and 222 that are movable relative to each other. In embodiments, the end effector 200 may include a proximal portion 212 having a first pin 213 and a distal portion 214. The end effector 200 may be actuated using a plurality of the cables 10 routed through proximal and distal portions 212 and 214 around their respective pulleys 212a, 212b, 214a, 214b, which are integrally formed as arms of the proximal and distal portions 212 and 214. In embodiments, the end effector 200, namely, the distal portion 214 and the jaws 220 and 222, may be articulated about the axis “A-A” to control a yaw angle of the end effector with respect to a longitudinal axis “X-X”. The distal portion 214 includes a second pin 215 with a pair of jaws 220 and 222 pivotably coupled to the second pin 215. The jaws 220 and 222 configured to pivot about an axis “B-B” defined by the second pin 215 allowing for controlling a pitch angle of the jaws 220 and 222 as well as opening and closing the jaws 220 and 222. The yaw, pitch, and jaw angles are controlled by adjusting the tension and/or length and direction (e.g., proximal or distal) of the cables 10. Thus, the end effector 200 may have three degrees of freedom, yaw, pitch, and jaw angle between jaws 220 and 222.

With reference to FIG. 2, the cable 10 also includes a coating 20. The coating 20 may also be applied to components of the instrument 150 that contact the cables 10 such as the pulleys 212a, 212b, 214a, 214b. The coating 20 lubricates the cable 10 as well as individual filaments 14 and the components of the instrument 150 to reduce frictional wear, increasing the hardness and the galling of wear surfaces.

The coating 20 may be formed using over-molding, extrusion, compression molding, spraying, electro-plating, electroless plating, or by vapor deposition processes to apply a coating material onto the cable 10. The material is applied into the interstitial spaces, crevices, and other surface textures inherent to multi-filament, twisted cables. The coating 20 may have a thickness from about 0.0005 mm to about 0.05 mm. The relative low thickness allows for the dimensional constraints on miniature, multi-filament cables used in minimally invasive device mechanisms and instruments such as the instrument 150 of FIGS. 3 and 4. In certain embodiments, the coating material may be any suitable hard and low friction material, such as tungsten disulfide (WS₂). WS₂ is a suitable material due to its wear capabilities and coefficient of friction. The coating 20 according to the present disclosure may have a static coefficient of friction of about 0.6 or less and a dynamic coefficient of friction of about 0.3 or less.

WS₂ provides an exceptional metallic molecular bond and adhesion to the cable substrate. The application process does not affect the base material or substrate which is a major advantage over most heat cured coatings that require binders and temperatures that can alter the temper and hardness of the base materials or electroplated coatings that can induce hydrogen embrittlement and usually require an oven tempering process to stress relive the base materials. WS₂ coating also provides exceptional chemical stability as well as corrosion and chemical resistance. This property allows the coating to withstand multiple sterilization and reprocessing cycles which may include extreme pH environments. Additionally, the WS₂ coating has an operating temperature of about 650° C., which provides a significant margin over autoclave temperatures, which may be up to about 137° C.

In addition, the coating 20 containing WS₂ is applied as a dry film that does not migrate or creep from the heat and pressures anticipated during use and/or sterilization or reprocessing cycles. During sterilization and reprocessing, the instrument 150 as well as its components is exposed to a variety of chemical cleaners, which may include alkaline and enzymatic detergents. As described above, chemical resistivity of WS₂ prevents chemical interactions or physical scrubbing of the coating 20 in the presence of these chemicals. In particular, the coating 20 is resistant to interaction with proteases, amylases, and lipases, which are used in enzymatic detergents. Furthermore, the coating 20 is also resistant to high pH of alkaline detergents, which may have a pH of 10 to 11. During reprocessing, the instrument 150 may also be lubricated using conventional lubricants, which may be water-based lubricants containing propylene glycol or mineral oil, etc. Chemical resistivity of WS₂ also provides protection from such lubricants due to its resistivity to mineral oil and alcohol interactions.

The WS₂ coating is also inert, non-toxic, and biocompatible for in-human device applications. The hardness of the WS₂ coating may be from about 35 on the Rockwell C hardness scale (HRC). Wear capabilities can be increased when combined with other materials or harder alloy base coatings to provide higher hardness attributes up to 70 HRC beneath WS₂ coating for additional wear protection. Suitable base alloy and coating materials may include but are not limited to: titanium nitride, titanium, nickel, nickel boron, nickel-polytetrafluoroethylene (PTFE), chromium, chromium nitride, chromium nickel, zirconium nitride, amorphous carbon, boron, boron carbide, molybdenum disulfide, molybdenum disulfide, molybdenum, graphite, silicone, zirconium oxide, and combinations thereof. Thus, the coating 20 can also include a hard material base surface that prevents accelerated wear and degradation caused by high sliding surface pressures between the twisted bundles 12 and the filaments 14 within the cables 10 as well as between the cable 10 and pulley surfaces. The materials and coatings used target the highest surface hardness viable for the optimal tensile and elongation properties of the bundles 12 and the filaments 14 of the cables 10 and pulleys.

To prevent galling, a nominal disparity in the surface hardness of the cable 10 and any mating pulleys or any other mating, bearing, constraining, sliding, manipulating components of the instrument 150 may be about 5 HRC or larger. The coating 20 may be used to reduce variation in the hardness of the two mating parts.

Because the cables are the more susceptible to fatigue and failure, it is desired the have the pulleys and all other components that interact with the cables, to be made with the lower surface hardness, for them to be the sacrificial wear components between the two mating parts. Thus, the cable 10 may have a higher coating surface hardness than the pulleys and other mating components.

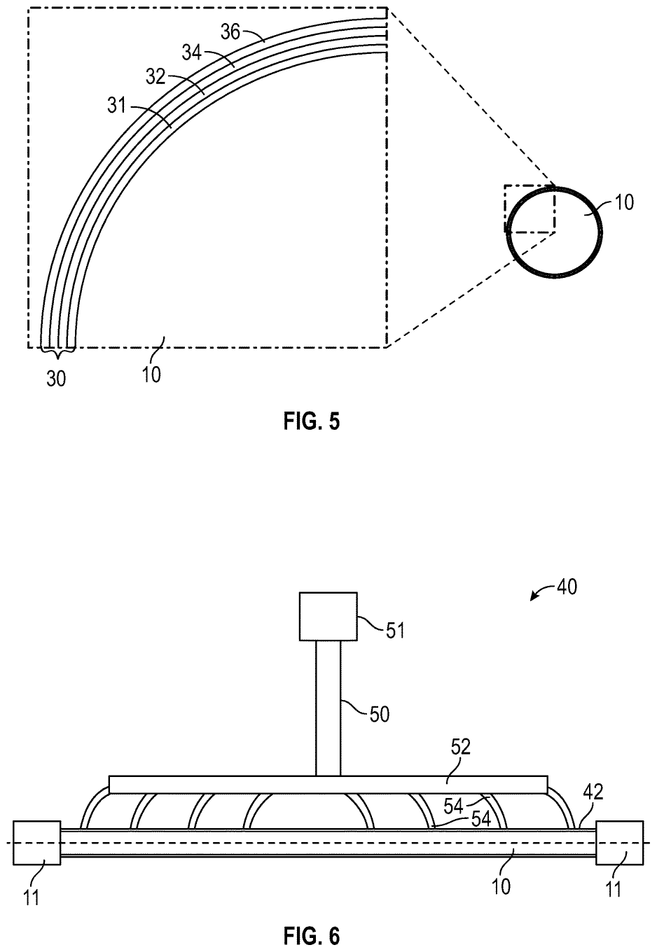

FIG. 5 shows a multilayer coating 30, which is a combination coating of multiple materials and includes a plurality (i.e., two or more) coatings each of which is formed from a different material. Like the coating 20, the coating 30 may be applied the cables 10 or and of the mating pulley and/or cable components. The coating 30 includes a base coating 31 formed from one or more hard and/or low friction coating material, which includes, but is not limited to, the following material and/or alloy combinations, titanium nitride, titanium, nickel, nickel boron, nickel-polytetrafluoroethylene (PTFE), chromium, chromium nitride, chromium nickel, zirconium nitride, amorphous carbon, boron, boron carbide, molybdenum disulfide, molybdenum, graphite, silicone, or zirconium oxide. The base coating 31 has a higher hardness attribute that the subsequent coating applied over the base coating 31, i.e., the first coating 32, which is formed from WS_{2.}

The first coating 32 is substantially similar to the coating 20 and is applied over the base coating 31. The first coating 32 may be formed by over-molding, extrusion, compression molding, spraying, electro-plating, electroless plating, or by vapor deposition processes to obtain thin and homogeneous thicknesses, which may be from about 0.0005 mm to about 0.05 mm. The combinations of alloy coatings also improves the hardness of the wear surfaces of the cables 10 or any of the mating pulley and/or cable components. The improved hardness of the first coating 32 improves the wear properties of the coated surfaces and provides a smoother overall surface finish and roughness of the drawn cables exterior surfaces to support reducing the surface frictions, surface porosity which results in reduced wear and improve the sliding efficiencies of the cables to any of their mating components.

The multilayer coating 30 also includes a second coating 34 formed from a polymer material. The second coating 34 is disposed over the first coating 32. The second coating 34 is formed from a hard polymer material and provides a lubricious, sacrificial wear surface to protect the cable 10 that should not creep or migrate away from the primary wear and bearing surfaces within a cable-pulley mechanism or assembly. The second coating 34 may be applied using over-molding, extrusion, compression molding, spraying, or vapor deposition processes and may have a thickness from about . 01 mm to about 0.2 mm. Suitable polymers for the second coating 34 may have a melting point of 140° C. or above to withstand the temperatures encountered during the autoclave process. The polymers also have exceptionally low friction, high heat deflection temperature (HDT) properties, and high-pressure velocity (PV) attributes. Suitable polymers include, but are not limited to, polytetrafluoroethylenes (PTFE), tetrafluoroethylenes (TFE), perfluoroalkoxy alkanes (PFA), fluorinated ethylene propylenes (FEP), polyimides, polyethylenimines (PEI), polyoxymethylenes (POM), polyether ether ketones (PEEK), copolymers, and combinations thereof. Glass or carbon fiber additives may also be added to improve the HDT or PV properties of the polymer-based second coating 34.

Additionally, the multilayer coating 30 includes a third coating 36 formed from a dry and/or solid and/or liquid lubricant, which include, but are not limited to silicone, petroleum, graphite, synthetic based greases or oils. The third coating 36 may also be applied using over-molding, extrusion, compression molding, spraying, or vapor deposition processes and may have a thickness from about 0.01 mm to about 0.2 mm. The third coating 36 provides a smoother overall surface finish and decreases the roughness of the exterior surfaces of the cable 10 to support reducing the surface frictions, surface porosity. The additional lubricity results in reduced wear and improves the sliding efficiencies of the cables 10 to any of their mating components.

The multilayer coating 30 provides protection for any of the base materials used in the instrument cables 10 and/or any of the mating pulley and/or cable components through extreme chemical environments and for the extreme temperatures of the autoclave steam sterilization cycles. These protective coatings also prevent or reduce the chemical and corrosive degradation anticipated from these processes, which help to maintain the functional efficiencies and extend the functional working life and the reliability of the surgical instrument cables 10.

In embodiments, only certain coatings 31, 32, 34, 36 may be used, i.e., one to three coatings may be skipped. Thus, the coating 31, 32, 34, 36 may be applied individually or if applied together, the coatings having a higher hardness are applied initially, with the coatings having a lower hardness being applied over the harder coating as described above with respect to FIG. 5. Thus, the coating 32 of WS₂ when applied, can be subsequently coated with the coating 34 and/or coating 36. However, if both coatings 34 and 36 are used, then the coating 34 is applied prior to the coating 36. Similarly, the coating 31, if applied, is applied prior to the coating 32. Accordingly, the coatings 31, 32, 34, 36 are sequentially layered as shown in FIG. 5 based on their relative hardness, with the hardest coating (e.g., coating 31) being applied first, and second hardest coating (e.g., coating 32) being applied second, etc.

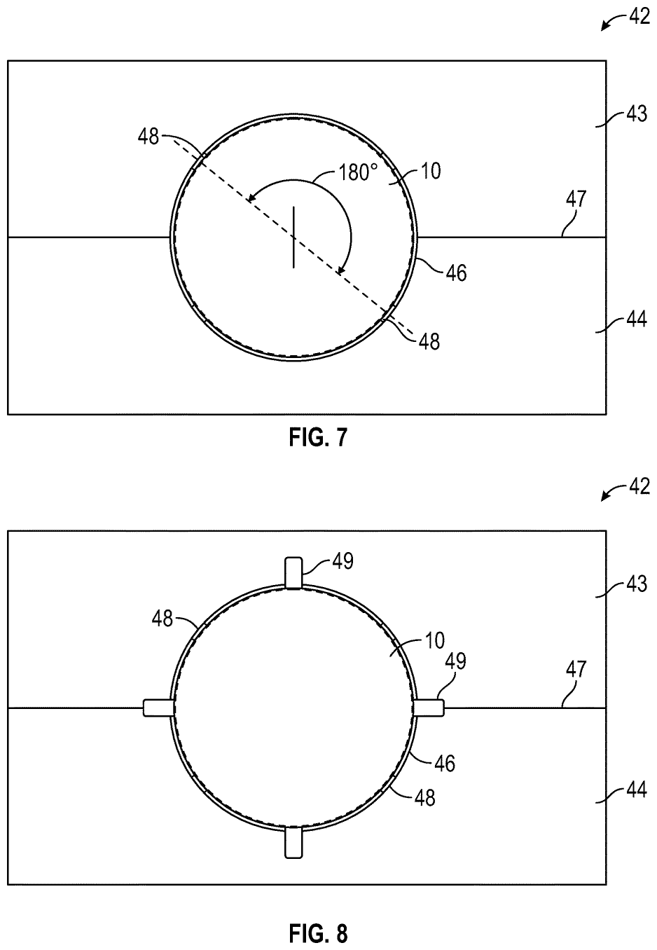

With reference to FIGS. 6-8, a coating system 40 may be used to form the second (i.e., polymer) coating 34 and/or third coating 36 of the multilayer coating 30. The coating system 40 may be used in any molding process, such as over-molding, injection molding, gravity molding, or extrusion processes. The coating system 40 includes a mold 42 having a first portion 43 and a second portion 44 defining a mold cavity 46 therein as shown in FIGS. 7 and 8. The mold cavity 46 has a substantially circular cross section to match the cross section of the cable 10 and has a slightly larger diameter than the diameter of the cable 10. The mold cavity 46 defines a mold parting line 47 at which the first and second portions 43 and 44 contact each other in a liquid-tight manner. In embodiments, the mold cavity 46 may be larger than the diameter of the cable 10 by a desired thickness of the coating being applied, e.g., from about 0.01 mm to about 0.2 mm. The mold portions 43 and 44 are opened and the cable 10 is loaded therein with the ferrules 11 extending outside the mold 42.

With reference to FIGS. 7 and 8, the mold cavity 46 may have a plurality of detents 48 disposed therein to constrain and center the cable 10 within the mold cavity 46. The detents 48 may run the entire length of the mold cavity 46. The detents 48 assist the material to flow around the cable 10 to properly fill the interstitial spaces within the cable and may protrude approximately 0.01 mm or more into the mold cavity 46. The detents 48 may be integrally formed with the first and second portions 43 and 44 and may be evenly spaced in a consistent pattern or may be staggered along the circumference of the mold cavity 46. In embodiments, each of the detents 48 may have a counterpart, diametrically opposite detent rib 48, e.g., 180° apart.

The detents 48 may be straight, i.e., parallel to a longitudinal axis defined by the mold cavity 46, or spiraled, to assist polymer material flow. The detents 48 may also be disposed in a consistent pattern or staggered. The detents 48 are configured to center the cable 10 within the mold cavity and may be in contact with the cable 10 or in a mild interference with the cable 10 and may be spaced about 0.01 mm or less from the cable 10.

With reference to FIG. 8, which shows another embodiment of the mold 42 having one or more channels 49, which are used to assist material flow during the over-molding process. The channels 49 may be disposed along the mold parting line 47 and/or in a plane perpendicular to the mold parting line 47 as shown in FIG. 8. Following the mold process, the material forms fins by filling the channels 49, which are trimmed post-process. Placing the channels 49 approximately 180° apart simplifies the trimming process. However, the channels 49 may be placed along any portion of the circumference of the mold cavity 46.

With reference to FIG. 6, the material for forming the second (i.e., polymer) coating 34 is provided to the mold 42 through a primary mold gate runner 50 from a source 51 of the material (e.g., a heated tank), which is coupled to a gate manifold 52 feeding one or more sub-gates 54, each of which, is in turn coupled to the mold 42 at corresponding multiple locations. This configuration allows for filling of the mold 42 in a consistent and uniform manner. The mold gating may include single, perpendicular, or axial gating. The number of sub-gates may increase in quantity based on the cable length to lower the gate pressures and optimize the over-mold fill of the interstitial spaces within the cable 10.

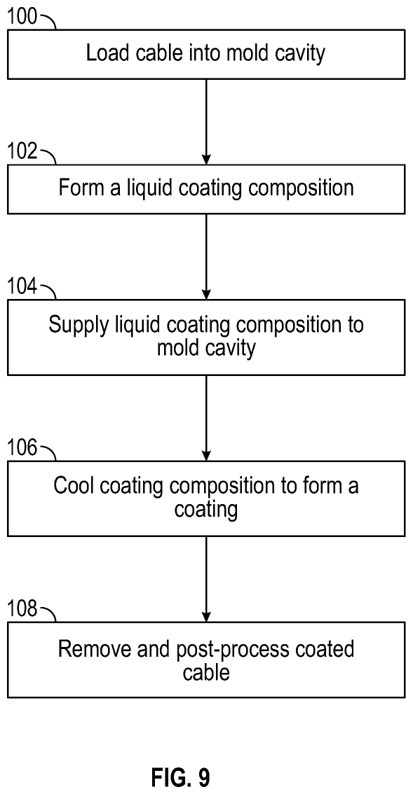

With reference to FIG. 9, a method of applying the second coating 34 is disclosed. At step 100, the cable 10 is loaded into the mold 42 by opening the first and second portions 43 and 44, placing the cable 10 into the mold cavity 46, and securing the mold 42 around the cable 10. At step 102, the coating material is melted to form a liquid composition suitable for the molding process and is supplied to the mold 42 through the gate manifold 52 at step 104. The flow of the liquid material during step 104 may be controlled by any suitable mechanisms until the liquid material has sufficiently entered the mold cavity 46. At step 106, the mold 42 is cooled to allow the second coating 34 to form. At step 108, the cable 10 along with the coating is removed and is post-processed to remove protrusions formed by the channels 49.

In embodiments where multiple coatings 34 and 36 of the multilayer coating 30 are applied, the method of FIG. 9 may be repeated to form each of the of the coatings 34 and 36. Thus, after forming the first coating 32, the coated cable 10 is processed again to form the second coating 34, etc. This may be possible by using materials for the first coating 32, that has a higher melting point, e.g., WS₂, than the melting point of the material of the second coating 34, e.g., polymers listed above. Thus, the material of the third coating 36, e.g., a grease or oil, has a lower melting point than the material of the second coating 34.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

1. A surgical instrument comprising:
a pulley rotatable about a pivot pin;
an end effector coupled to the pivot pin; and
a cable wound at least partially around the pulley and configured to actuate the end effector, wherein the cable includes a first coating having tungsten disulfide.

2. The surgical instrument according to claim 1, wherein the first coating has a hardness of about 35 on a Rockwell C hardness scale (HRC).

3. The surgical instrument according to claim 1, wherein the first coating is applied over a base coating disposed on the cable applied, wherein the base coating has a hardness above 35 HRC.

4. The surgical instrument according to claim 3, the base coating is from a material selected from the group consisting of titanium nitride, titanium, nickel, nickel boron, nickel-polytetrafluoroethylene, chromium, chromium nitride, chromium nickel, zirconium nitride, amorphous carbon, boron, boron carbide, molybdenum disulfide, molybdenum disulfide, molybdenum, graphite, silicone, and zirconium oxide.

5. The surgical instrument according to claim 3, wherein the cable includes a second coating disposed over the first coating, the second coating including a polymer.

6. The surgical instrument according to claim 5, wherein the polymer is selected from the group consisting of polytetrafluoroethylenes, tetrafluoroethylenes, perfluoroalkoxy alkanes, fluorinated ethylene propylenes, polyimides, polyethylenimines, polyoxymethylenes, polyether ether ketones, copolymers, and combinations thereof.

7. The surgical instrument according to claim 5, wherein the cable further includes a third coating disposed over the second coating, the third coating including at least one of a wax-based, paraffin-based, a grease-based, or an oil-based lubricant.

8. The surgical instrument according to claim 1, wherein at least one of the pulley or the pivot pin includes the first coating having tungsten disulfide.

9. The surgical instrument according to claim 1, wherein at least one of the cable, the pulley, or the pivot pin includes a multilayer coating having the first coating as one of layers of the multilayer coating.

10. The surgical instrument according to claim 1, wherein the first coating has a static coefficient of friction of about 0.6 or less and a dynamic coefficient of friction of about 0.3 or less.

11. The surgical instrument according to claim 1, wherein the first coating has a homogenous thickness from about 0.0005 mm to about 0.05 mm.

12. A system for coating a surgical instrument cable, the system comprising:
a mold including a first portion and a second portion defining a mold cavity configured to receive the surgical instrument cable, wherein the mold cavity includes a plurality of detents running along a length of the mold cavity;
a source of a liquid material for forming a coating on the surgical instrument cable inside the mold cavity; and
a gate manifold configured to distribute the liquid material through the mold cavity to form the coating on the surgical instrument cable.

13. The system according to claim 12, wherein each detent of the plurality of detents is disposed in a parallel or spiral configuration relative to other detents along the length of the mold cavity.

14. The system according to claim 12, wherein the first portion and the second portion contact each other along a mold parting line and the mold cavity further includes a first pair of channels disposed along the mold parting line and a second pair of channels disposed in a plane perpendicular to the mold parting line.

15. The system according to claim 12, wherein the coating includes a polymer.

16. The system according to claim 15, wherein the polymer is selected from the group consisting of polytetrafluoroethylenes, tetrafluoroethylenes, perfluoroalkoxy alkanes, fluorinated ethylene propylenes, polyimides, polyethylenimines, polyoxymethylenes, polyether ether ketones, copolymers, and combinations thereof

17. A method for coating a surgical instrument cable, the method comprising:
placing a surgical instrument cable into a mold including a first portion and a second portion defining a mold cavity configured to receive the surgical instrument cable, wherein the mold cavity includes a plurality of detents running along a length of the mold cavity; and
supplying a liquid material to the mold cavity to form a coating on the surgical instrument cable.

18. The method according to claim 17, further comprising:
placing the surgical instrument cable having a first coating into the mold; and
supplying a second liquid material to the mold cavity to form a second coating over the first coating, wherein the second coating includes a polymer.

19. The method according to claim 18, wherein the polymer is selected from the group consisting of polytetrafluoroethylenes, tetrafluoroethylenes, perfluoroalkoxy alkanes, fluorinated ethylene propylenes, polyimides, polyethylenimines, polyoxymethylenes, polyether ether ketones, copolymers, and combinations thereof.

20. The method according to claim 18, further comprising:
placing the surgical instrument cable having the first coating and the second coating into the mold; and
supplying a third liquid material to the mold cavity to form a third coating over the second coating, wherein the third coating includes at least one of a wax-based, paraffin-based, a grease-based, or an oil-based lubricant.
